(19) United States
(12) Patent Application Publication
(10) Pub. No.: US 2021/0316023 A1
(43) Pub. Date: Oct. 14, 2021

(54) DUAL-PURPOSE STERILIZATION BOX

(71) Applicant: HORNIX OPTICAL TECHNOLOGY INC., Taoyuan City (TW)

(72) Inventor: Ying-Cheng LIN, TAOYUAN CITY (TW)

(21) Appl. No.: 17/170,512

(22) Filed: Feb. 8, 2021

(30) Foreign Application Priority Data
Apr. 8, 2020 (TW) 109204038

(51) Int. Cl. A61L 2/10; A61L 2/26

(52) U.S. Cl. CPC A61L 2/10; A61L 2/26; A61L 2202/11; A61L 2202/121; A61L 2202/122

(57) ABSTRACT

A dual-purpose sterilization box includes an outer box set provided with sterilization light source elements, an inner box placed in a housing space inside the outer box set and provided with through holes on side walls thereof corresponding to the sterilization light source elements so that the sterilization light source elements can emit sterilization light into the housing space around 360 degrees to sterilize items placed in the housing space. A light guide box for storing food can be placed in the housing space, so that sterilization light source elements in the outer box set can emit sterilization light to an optical structure of light incident portion of the light guide box and a light emitting surface of the light guide box can project the incident sterilization light into the sterilization space in the light guide box through 360 degrees to sterilize the storage food.

### BACKGROUND OF THE INVENTION

### 1. Field of the Invention:

The present invention relates to sterilization boxes and more particularly, to a dual-purpose sterilization box, which use a sterilization light source to sterilize non-edible objects, or a light guide box for storing food to sterilize food.

### 2. Description of the Related Art:

Modern people pay more and more attention to food safety. In addition to keeping food fresh, the tableware and utensils used must also be cleaned. Therefore, there are sterilization devices and a variety of storage containers for related tableware and utensils on the market. However, consumers not only have to purchase sterilization devices and storage containers at the same time, but also take up space. Moreover, the current common sterilization devices use heating liquid (such as water) to generate high-temperature steam to sterilize and dry items to ensure the safety and hygiene of items. However, this design is not only too complicated in structure and operation, but also needs to confirm the amount of liquid to be added every time it is used, which causes trouble in use. The current food storage container design includes a container body and a detachable cover. When the container is repeatedly opened to take out the food in the container body, the outside air will enter the inside of the container body every time the food is taken. If the food is not used or consumed in a short time, it will easily deteriorate.

### SUMMARY OF THE INVENTION

The present invention has been accomplished to provide a dual-purpose sterilization box, which solves the inability to combine the sterilization function of an existing disinfection device and a fresh-keeping container, so that the user needs to purchase an existing disinfection device and a fresh-keeping container separately, which increases the consumption cost and also takes up storage space, and which eliminates the drawbacks that the existing disinfection device has complex structure and operation and the outside air will enter the inside of the container body to deteriorate the storage food every time the food is taken out of the container body of the fresh-keeping container.

It is therefore the main object of the present invention to provide a dual-purpose sterilization box, which uses a sterilization light to sterilize non-edible objects in housing space to ensure the safety of the objects, and allows the user to add a light guide box made of UVC band light guide material that can store food according to requirements, so that a comprehensive light source can be applied to sterilize the food in the sterilization space in a 360-degree space through an optical structure of an optical design.

To achieve this and other objects of the present invention, a dual-purpose sterilization box comprises an outer box set and a sterilization light source module. The outer box set comprises an inner box and a housing space. The housing space is defined in the inner box. The inner box has a plurality of through holes on side walls thereof on different sides of the housing space. The sterilization light source module is set in the outer box set, comprising a plurality of sterilization light source elements corresponding to the through holes of the inner box respectively and adapted for emitting a sterilization light into the housing space.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an oblique top elevation of a dual-purpose sterilization box in an opened status in accordance with the present invention.

FIG. 2 is an exploded view of the dual-purpose sterilization box shown in FIG. 1.

FIG. 3 is a sectional view of the dual-purpose sterilization box in accordance with the present invention.

FIG. 4 is a sectional elevation, in an enlarged scale, of a part of the light guide box.

FIG. 5 is an enlarged view of a part of FIG. 3.

FIG. 6 is an exploded view of an alternate form of the present invention.

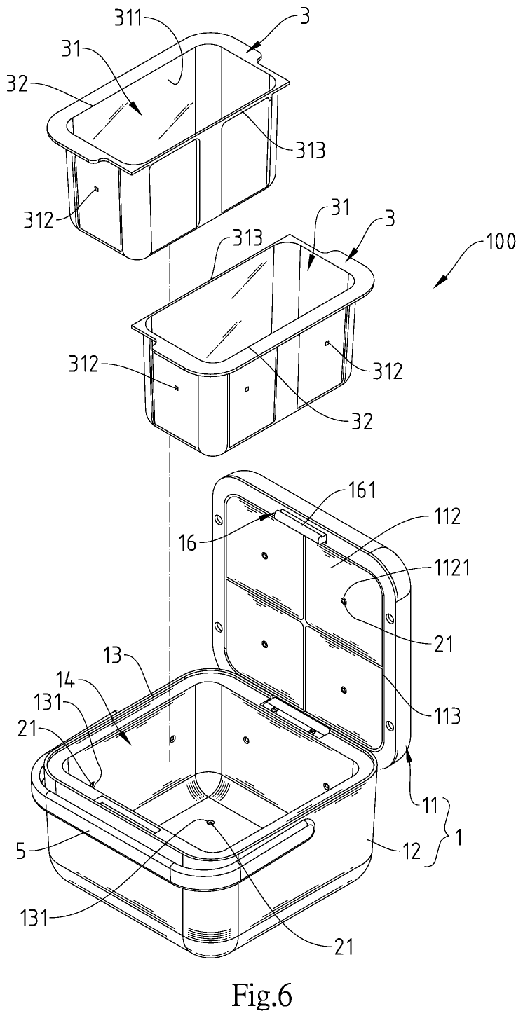

### DETAILED DESCRIPTION OF THE PREFERRED EMBODIMENT

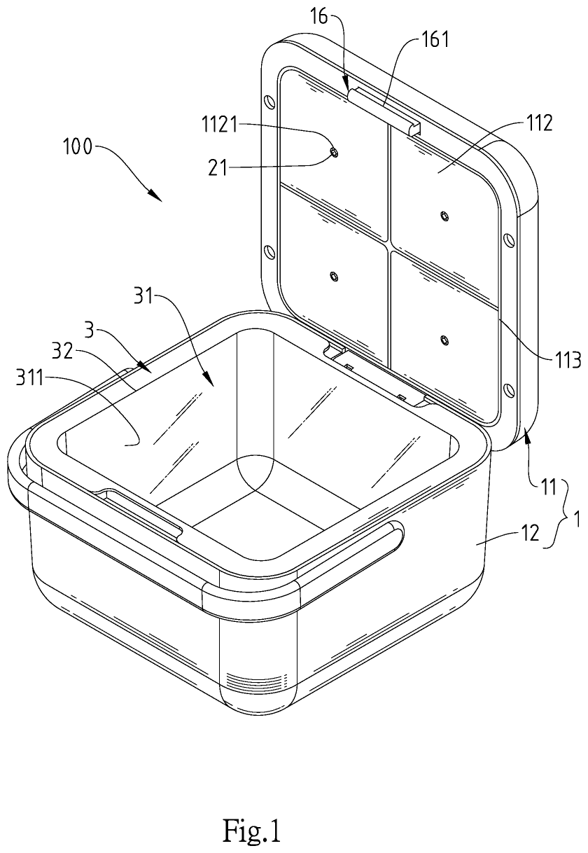

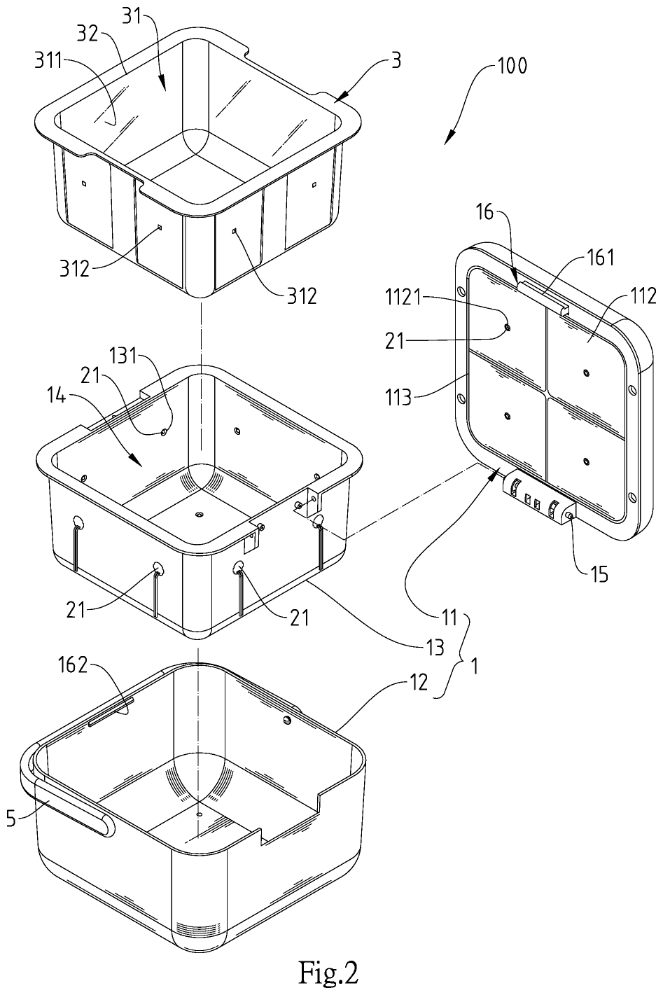

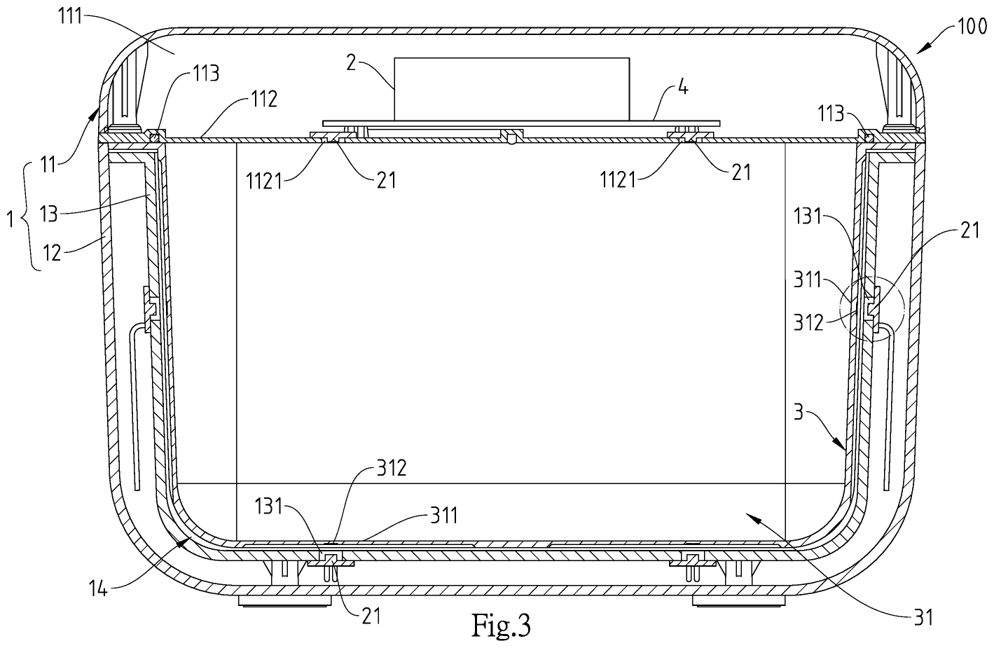

Referring to FIGS. 1, 2 and 3, a dual-purpose sterilization box 100 comprises an outer box set 1, a sterilization light source module 2, a light guide box 3 and a circuit controller 4.

The sterilization light source module 2 is set in the outer box set 1. The circuit controller 4 is electrically connected to the sterilization light source module 2. The circuit controller 4 is used to control the sterilization light source module 2 to emit sterilization light. The dual-purpose sterilization box 100 further comprises a handle 5, which is set on the outer box set 1 and can provide users with convenient portability.

The outer box set 1 comprises an upper box 11, a lower box 12 and an inner box 13. The outer box set 1 defines therein a housing space 14. The outer box set 1 has through holes on the side walls on different sides of the housing space 14. Since the inner box 13 is arranged in the lower box 12, the housing space 14 is located in the inner box 13, so the side walls of the housing space 14 of the inner box 13 are respectively provided with through holes, namely, the lower through holes 131.

The sterilization light source module 2 comprises a plurality of sterilization light source elements 21 set between the lower box 12 and the inner box 13 to aim at the lower through holes 131 of the inner box 13 respectively, so that the sterilization light source elements 21 can emit sterilization light source into the housing space 14. In this embodiment, the inner box 13 has lower through holes 131 on each side wall of the housing space 14, and the bottom wall of the housing space 14 is also provided with lower through holes 131. Furthermore, each lower through hole 131 corresponds to one respective sterilization light source element 21.

The upper box 11 is pivotally connected to one side of the lower box 12 and the inner box 13 with a pivot member 15. By means of the pivot member 15, the upper box 11 can be covered on the housing space 14 or opened away from the housing space 14. The upper box 11, the lower box 12, and the inner box 13 have a matching opening and closing device 16 on the other side remote from the pivot member 15. In the closing operation of the matching opening and closing device 16, the upper box 11 is covered on the housing space 14 by the rotation of the pivot member 15. In the opening operation of the matching opening and closing device 16, the upper box 11 is opened by the rotation of the pivot member 15 and stayed away from the housing space 14. Specifically, the upper box 11 has an operating member (not shown). The matching opening and closing device 16 comprises a first engaging member 161 and a second engaging member 162. The operating member is connected to the first engaging member 161. The first engaging member 161 is located on one side of a cover panel 112 of the upper box 11. The second engaging member 162 is located on an inner side of the lower box 12. The first engaging member 161 and the second engaging member 162 are designed to cooperate. The first engaging member 161 can be engaged with the second engaging member 162 through the operation of the operating member to form a locked state, or the first engaging member 161 can be disconnected from the second engaging member 162 through the operation of the operating member to form an unlocked state. The upper box 11 comprises an installation chamber 111 and the aforesaid cover panel 112. The cover panel 112 covers the installation chamber 111. The cover panel 112 has through holes, namely, the upper through hole 1121. The installation chamber 111 has set therein sterilization light source elements 21 respectively aimed at the upper through holes 1121 of the cover panel 112, so that the sterilization light source elements 21 can emit sterilization light into the housing space 14. The housing space 14 can accommodate non-edible objects, such as tableware, utensils, baby bottles or any objects that need to be sterilized. The sterilization light source elements 21 are ultraviolet light source elements. The ultraviolet light emitted by the sterilization light source elements 21 can damage microorganisms such as bacteria, viruses, spores and other pathogens and destroy the function of nucleic acids, so as to achieve the purpose of sterilization and ensure the safety of obj ects.

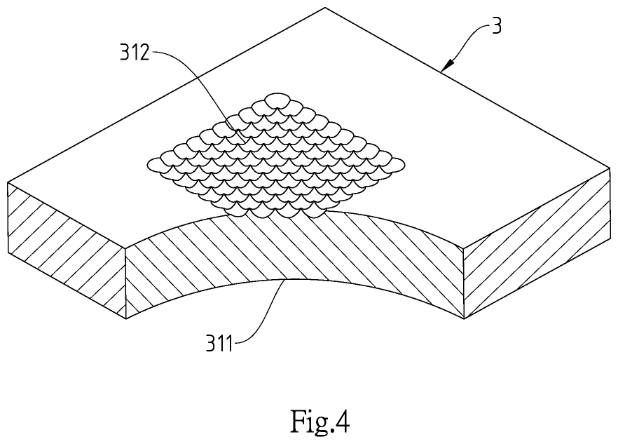

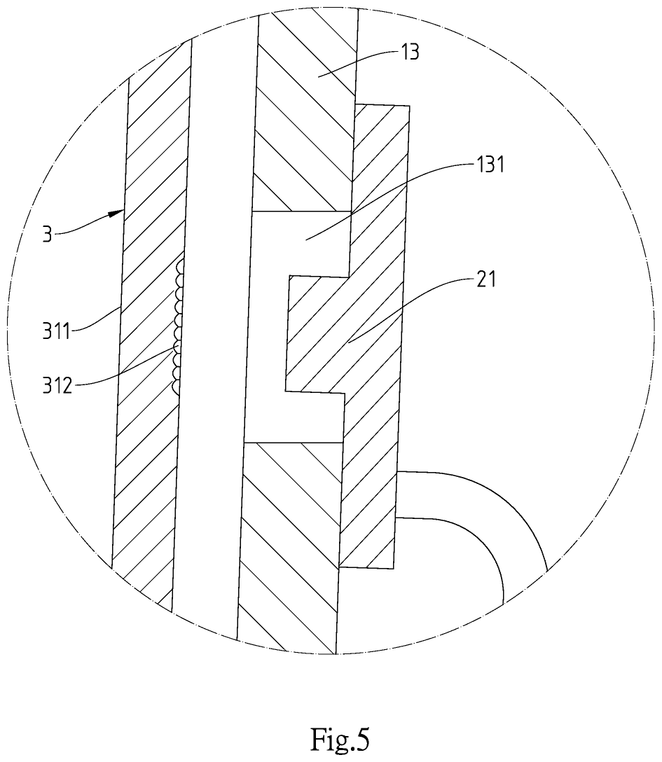

In addition, food can be sterilized. The light guide box 3 can be placed in the housing space 14. The light guide box 3 has a sterilization space 31 and an opening 32. The sterilization space 31 is disposed in communication with the opening 32. The light guide box 3 has a light emitting surface 311 formed on the side wall near the periphery of the sterilization space 31, and a light incident portion 312 is provided on the side wall surface away from the light emitting surface 311. The light incident portion 312 is facing the lower through holes 131 of the outer box set 1. The sterilization light emitted by the sterilization light source elements 21 passes through the lower through holes 131 to the light incident portion 312, and then is projected from the light emitting surface 311 toward the sterilization space 31. As shown in FIGS. 4 and 5, the light guide box 3 is made of UVC band light guide and transparent materials. The light incident portion 312 is an optical structure of optical design, such as microstructure. When each sterilization light source element 21 projects the sterilization light directly to the microstructure through the lower through holes 131, the sterilization light is refracted at multiple angles through the microstructure, so that the light emitting surface 311 can project the sterilization light in the form of a 360 degrees surface light source into the sterilization space 31. The sterilization light source elements 21 located in the upper box 11 directly projects the sterilization light into the sterilization space 31 through the upper through holes 1121.

After the food is placed in the sterilization space 31 through the opening 32, the upper box 11 is covered on the sterilization space 31 through the rotation of the pivot member 15, and the first engaging member 161 is fastened to the second engaging member 162 through the operation of the operating member to form a locked state. The circuit controller 4 is used to control the operation of the sterilization light source module 2. The upper, lower and peripheral sides of the outer box set 1 are all equipped with the sterilization light source elements 21. When all the sterilization light source elements 21 emit sterilization light toward the sterilization space 31, the effect of comprehensive sterilization of the food in the sterilization space 31 can be achieved to ensure the safety of the food and avoid food spoilage.

As shown in FIG. 7, the invention can accommodate more than one light guide box 3 in the housing space 14 of the outer box set 1. In this embodiment, two light guide boxes 3 are installed as an example. The two light guide boxes 3 each have an abutting portion 313 on the outer side, and the abutting portions 313 of the two light guide boxes 3 are matched, and the abutting portions 313 of the two light guide boxes 3 are used to abut against each other. The cover panel 112 of the upper box 11 is further provided with a sealing element 113, such as a sealing strip. The sealing element 113 is designed according to the quantity of the light guide box 3. The sealing element 113 corresponds to the upper inner edge of the sterilization space 31 of the light guide box 3. The sealing element 113 can completely block the outside air from entering the sterilization space 31. In this way, the invention can solve the problem of the existing integrally-formed compartmentalized fresh-keeping containers that whenever the user opens the lid to take the food out of the container body, the air will enter the fresh-keeping container again, which not only easily deteriorates the food, but also cannot be taken out, replaced or cleaned in a single box.

What the invention claimed is:

1. A dual-purpose sterilization box, comprising:
an outer box set comprising an inner box and a housing space, said housing space defined in said inner box, said inner box having a plurality of through holes on side walls thereof on different sides of said housing space; and
a sterilization light source module set in said outer box set, said sterilization light source module comprising a plurality of sterilization light source elements corresponding to said through holes of said inner box respectively and adapted for emitting a sterilization light into said housing space.

2. The dual-purpose sterilization box as claimed in claim 1, further comprising at least one light guide box for placing in said housing space, each said light guide box comprising a sterilization space, an opening in communication with said sterilization space, a light emitting surface formed on a side wall thereof near the periphery of said sterilization space and a light incident portion formed on a side wall surface thereof far away from said light emitting surface, said light incident portion facing said through holes of said inner box for allowing said sterilization light source elements to emit said sterilization light through said through holes onto said light incident portion, said light emitting surface being adapted to project said sterilization light into said sterilization space.

3. The dual-purpose sterilization box as claimed in claim 2, wherein said light guide box is made of a UVC band light guide and transparent material; said light incident portion is a microstructure; each said sterilization light source element is configured to emit said sterilization light to said microstructure.

4. The dual-purpose sterilization box as claimed in claim 2, wherein the number of said at least one light guide box is two, each said light guide box comprising an abutting portion located on an outer side thereof, the two said light guide boxes being arranged side by side with the abutting portions thereof abutted against each other.

5. The dual-purpose sterilization box as claimed in claim 1, wherein each said sterilization light source element is a ultraviolet light source element.

6. The dual-purpose sterilization box as claimed in claim 1, further comprising a circuit controller set in outer box set and electrically connected to said sterilization light source module for controlling said sterilization light source module to emit said sterilization light.

7. The dual-purpose sterilization box as claimed in claim 1, wherein said outer box set further comprises an upper box and a lower box; said inner box is placed in said lower box; said sterilization light source elements of said sterilization light source module are arranged between said lower box and said inner box.

8. The dual-purpose sterilization box as claimed in claim 7, wherein said upper box comprises an installation chamber and a cover panel covering said installation chamber, said cover panel comprising a plurality of through holes; said sterilization light source module further comprises a plurality of sterilization light source elements mounted in said installation chamber corresponding to the said through holes of said cover panel for emitting sterilization light into said housing space.

9. The dual-purpose sterilization box as claimed in claim 7, wherein said upper box is pivotally connected to one side of said lower box and said inner box with a pivot member, so that said upper box is turnable with said pivot member to cover said housing space or to open from said housing space.

10. The dual-purpose sterilization box as claimed in claim 7, wherein said upper box, said lower box and said inner box have a matching opening and closing device provided on an opposite side thereof remote from said pivot member, so that in the closing operation of said matching opening and closing device, said upper box is covered on said housing space by the rotation of said pivot member; in the opening operation of said matching opening and closing device, said upper box is opened by the rotation of said pivot member and stayed away from said housing space.
